(19) United States
(12) Patent Application Publication
(10) Pub. No.: US 2026/0128155 A1
(43) Pub. Date: May 7, 2026

(54) WELLNESS PRACTICE RECOMMENDATION

(71) Applicant: Anastasia Filipenko, Dubai (AE)

(72) Inventor: Anastasia Filipenko, Dubai (AE)

(21) Appl. No.: 19/376,919

(22) Filed: Nov. 1, 2025

Related U.S. Application Data
(60) Provisional application No. 63/715,519, filed on Nov. 2, 2024.

(51) Int. Cl. G16H 20/70; G06F 3/0485; G06F 3/0488; G16H 40/67

(52) U.S. Cl. CPC G16H 20/70; G06F 3/0485; G06F 3/0488; G16H 40/67

(57) ABSTRACT

A system and method for generating personalized wellness recommendations in real time, based on a user's emotional state, contextual situation, and interaction history. The system leverages a large language model (LLM) integrated with an emotion ontology based on the Wheel of Emotion to analyze both user-submitted input and historical behavior. In some embodiments, the system extracts emotional and situational cues from natural language input and recommends wellness practices that match specified preferences including emotional impact, guidance level, and duration. A fallback prioritization logic is applied to ensure relevant results even when exact matches are unavailable. The system may also generate a compassionate message aligned with the user's inferred emotional state and return structured output. In automated embodiments, the system selects a weighted mix of familiar and exploratory content. The architecture includes preference drift tracking, confidence-based filtering, and memory-optimized re-ranking to improve emotional targeting and user engagement.

### CROSS REFERENCE TO RELATED APPLICATIONS

This application claims the benefit of U.S. Provisional Patent Application No. 63/715,519, filed on Nov. 2, 2024, and titled “System for Wellness Practice Recommendation Using Large Language Models and Method Thereof,” the entire disclosure of which is incorporated herein by reference.

### FIELD OF THE INVENTION

The present invention relates to the field of artificial intelligence (AI)-based wellness applications, and more particularly to a system for wellness practice recommendation using large language models and a method thereof.

### BACKGROUND OF THE INVENTION

Conventional wellness applications, such as Headspace and Calm, offer personalized meditation and mindfulness recommendations based on user inputs during on-boarding, meditation history, and predefined practice pathways. These applications use algorithms that categorize users' preferences based on factors including practice type, duration, and popularity. However, they lack in providing dynamic, real-time personalization that adapts to the user's evolving emotional state or specific feedback.

Additional drawbacks associated with similar applications are as follows: While existing wellness applications do offer personalized recommendations, the level of personalization might not be as nuanced as suggested. Their algorithms primarily use basic data points e.g., meditation history, session duration, rather than more sophisticated inputs like real-time emotional state or detailed psychological profiling. For example, when a user selects a practice, future recommendations are often derived from preset categories, which may not evolve with the user's emotional or mental state over time. This one-size-fits-all approach can lead to disengagement, as users may feel that the content lacks personal relevance. As a result, the user engagement diminishes, especially when the app fails to provide interactive feedback that adjusts in real time based on the user's emotional responses.

Further, these apps often rely on predefined practice pathways and user segmentation rather than dynamically responding to changing user states. For example, a user might receive recommendations based on their general preferences, but the app might not adapt immediately if the user's stress or mood changes throughout the day. The personalization offered by these conventional systems is surface-level, typically confined to the initial user inputs without considering the user's present emotional states. These systems do not incorporate user feedback about the emotional impact of past practices, the influence of specific meditation guides or authors, or preferences as they evolve over time. This lack of emotional responsiveness can result in generic recommendations, thus reducing the effectiveness of the wellness experience for the user.

Although some applications use machine learning, many of the recommendations are still driven by simpler logic-based algorithms or user journey frameworks. This means that they may not leverage complex AI models that could provide deeper personalization.

Additionally, users are often overwhelmed by the abundance of available options within these wellness apps. This leads to decision fatigue, as the burden of manually selecting from long lists of meditation practices can be both time-consuming and frustrating. This is especially problematic for users seeking immediate emotional relief or mindfulness guidance, as the process of finding suitable content detracts from the overall experience.

Therefore, there is a need for a solution that integrates a real-time emotional analysis system using large language models while allowing for dynamic adaptation and real-time, personalized recommendations of wellness and meditation practices. Further, by continuously assessing the user's emotional feedback, a system should facilitate offering recommendations based on user's current emotional state, reducing decision fatigue, and enhancing user engagement.

### BRIEF SUMMARY OF THE INVENTION

It is an object of the present invention to provide a wellness recommendation system that dynamically adapts to a user's emotional state using real-time semantic interpretation performed by a large language model (LLM).

It is another object of the present invention to provide personalized meditation recommendations based on multiple attributes, including emotional state, duration availability, wellness goals and guidance preferences, while continuously refining the system through user feedback.

It is yet another object of the present invention to reduce decision fatigue by automating wellness recommendations based on historical data and emotional patterns and user preferences, without requiring the user to choose from a vast array of content.

It is an object of the present invention to improve the accuracy and relevance of wellness recommendations over time by recording and analyzing user activity, including feedback and meditation preferences, to train the system for future interactions. In some embodiments, a system can be configured to additionally incorporate a feedback module and historical database to record user interactions such as skips, completions, and satisfaction ratings. A personalized recommendation module uses this historical data to generate future suggestions without requiring new user input, thus enabling improved engagement and emotional alignment over time.

It is another object of the invention to map natural language input into structured emotional categories using the Wheel of Emotion framework, and to use this mapping to guide the selection of relevant wellness practices from a tagged database.

It is another object of the present invention to provide personalized meditation recommendations based on multiple attributes, including emotional state, duration availability, wellness goals and guidance preferences, while continuously refining the system through user feedback.

Embodiments of the invention disclosed here need not rely on static keyword matching or predefined rule-based flows. Some embodiments of the systems disclosed here leverage a large language model with context-aware emotion detection to dynamically personalize recommendations in real-time. In some embodiments, a fixed prioritization logic and temporal adjustment mechanism further improve decision latency and user engagement by reducing cognitive burden on the user. One aspect of the invention is directed to a method for providing meditation recommendations. In one embodiment, the method involves receiving, via a sign-up module, user profile data and storing the user profile data in a user database; receiving, via an input module, request data comprising user text or voice describing a current emotional state and an available time duration; accessing a meditation database storing records tagged with at least one emotion category from a Wheel-of-Emotion framework, a desired outcome, a guidance level, and a duration. The method can further include applying, by an LLM engine, an input analysis module configured with an NLP engine and the Wheel-of-Emotion framework to the request data to produce an emotional-state classification; filtering, by a meditation matching module, the meditation records to those whose stored emotion tag equals the emotional-state classification; applying, by the meditation matching module, a fixed order of precedence: current emotional state, then desired outcome, then guidance level, then available time; and performing temporal adjustment, by the meditation matching module, by first selecting meditations within a threshold of the available time, then-if available-selecting shorter alternatives, and then-if available selecting meditations closest to but exceeding the available time.

The method can also involve presenting, via a graphical user interface (GUI), a compassionate message and multiple recommendation cards each with at least a title, and Play and Try again controls; and replacing, by the GUI, a previously presented set of the recommendation cards with an updated set of the recommendation cards in the same view when the LLM engine outputs updated recommendation data. The method can further include, in an automated mode, generating, by a personalized recommendation module, recommendations using stored user interactions in a historical database, including a mix of familiar and exploratory meditations according to predetermined percentages; recording, by a feedback module, skips, selections, completions, and ratings in the historical database and providing the recorded feedback to the LLM engine; and responsive to any of the following events: (a) a change to the available time, (b) a clarification of the emotional state, (c) a Try again request, or (d) a skip input received via the GUI, automatically, during the same user session, recomputing, by the LLM engine, recommendations by re-applying the fixed order of precedence and the temporal-adjustment steps, and updating the recommendations presented on the GUI.

In some embodiments, the stored records are further tagged with a meditation name, a meditation description, an emotional impact, and associated wellness goals. In certain embodiments, the method can further comprise in the automated mode generating, by the personalized recommendation module without real-time user input, recommendations based on: the user's previous emotional states within a predetermined time period, the user's interests, the user's preference for guidance, the user's wellness goals, and other user-specific parameters derived from the historical database or user data.

In one embodiment, the method can further involve receiving, by the GUI, a user swipe-based interaction to allow the user to indicate interest or disinterest in personalized meditation recommendations; and recording, by an activity tracking module, user responses to the personalized recommendations in the historical database for future analysis. In some embodiments, the method can further include processing the request data, by the LLM engine, using natural language processing to extract the contextual input phrases such as the attributes for recommendation generation.

Another aspect of the invention is concerned with a meditation recommendation system having a sign-up module to receive user profile data; a user database configured to store the user profile data; an input module to receive request data comprising user text or voice describing a current emotional state and an available time; and a meditation database storing records tagged with at least one emotion category from a Wheel-of-Emotion taxonomy, a desired outcome, a guidance level, and a duration. The system can further include an LLM engine having an input analysis module configured to apply an NLP engine and the Wheel-of-Emotion taxonomy to the request data to produce an emotional-state classification; and a meditation matching module configured to: filter meditations to those whose stored emotion tag equals the emotional-state classification; apply a fixed order of precedence: current emotional state, then desired outcome, then guidance level, then available time, and perform temporal adjustment by first selecting meditations within a threshold of the available time, then—if available—shorter alternatives, and then—if available—meditations closest to but exceeding the available time. The system can further include a graphical user interface (“GUI”) configured to present a compassionate message and multiple recommendation cards each with at least a title, guidance level, duration, and Play and Try again controls; wherein the GUI replaces a previously presented set of the recommendation cards with an updated set of the recommendation cards in the same view when the LLM engine outputs updated recommendation data. The system can further include a historical database; a personalized recommendation module configured to generate automated recommendations using user interactions stored in the historical database; and a feedback module configured to record skips, selections, completions, and ratings in the historical database and to provide the feedback to the LLM engine. The system can be configured wherein, responsive to any of the following events (a) a change to the available time, (b) a clarification of the emotional state, (c) a Try again request, or (d) a skip input received via the GUI, the LLM engine automatically, during the same user session, recomputes recommendations by re-applying the prioritization order and the temporal-adjustment steps and updates the recommendations presented on the GUI.

In one embodiment, the records stored in the meditation database are further tagged with an emotional impact, a practice duration, and associated wellness goals. In some embodiments, the personalized recommendation module is further configured to generate recommendations based on a mix of familiar and exploratory meditations according to predetermined percentages. In certain embodiments, the LLM is further configured to analyze the historical database, and the user data; and to automatically generate personalized meditation recommendations without real-time user input based on: the user's previous emotional states within a predetermined time period, the user's interests, the user's preference for guidance, the user's wellness goals, and other user-specific parameters derived from the historical database or user data.

In one embodiment, the GUI can be further configured to facilitate swipe-based interaction by the user to indicate interest or disinterest in personalized meditation recommendations; and the system further comprising an activity tracking module configured to record user responses to the personalized meditation recommendations in the historical database for future analysis. In some embodiments, the input analysis module is further configured to process the request data using natural language processing to extract the contextual input phrases such as the attributes for recommendation generation.

### BRIEF DESCRIPTION OF DRAWINGS

The present invention is clearly understandable to those of ordinary skill in the art when descriptions of exemplary embodiments thereof are read with reference to the accompanying drawings.

FIG. 1 is a high-level block diagram of a wellness recommendation system according to an embodiment of the present invention.

FIG. 2A depicts user interfaces (UIs) of a sign-up module, of the system of FIG. 1, for collecting user data.

FIG. 2B depicts UIs of an input module, of the system of FIG. 1, for collecting meditation request data.

FIG. 3 is a block diagram of a large language model (LLM) engine of the system of FIG. 1.

FIG. 4 is a block flow diagram of an input analysis module of the LLM of FIG. 3.

FIG. 5 is a block flow diagram of a meditation matching module of the LLM of FIG. 3.

FIG. 6 is another block flow diagram of the meditation matching module of the LLM of FIG. 3.

FIG. 7 is a block flow diagram of a personalized meditation recommendation module of the system of FIG. 1.

FIG. 8A-8B are a flow chart of a method according to one embodiment of the invention.

### DETAILED DESCRIPTION OF THE INVENTION

The term “artificial intelligence” or AI used in this disclosure typically refers to “machine intelligence” that includes a computer model, algorithm or simulation of human intelligence processes by machines, such as computer systems to learn, predict, analyze and provide actionable insight, and/or control actuators. In exemplary aspects, the AI includes “conversational AI” or “conversational AI model.” The AI may be a machine learning algorithm, wherein the machine learning algorithm may include a trained machine learning algorithm. Typically, the machine learning algorithm may be trained using supervised, semi-supervised, unsupervised or reinforcement learning techniques which includes neural networks and support vector machines.

In an exemplary embodiment, a wellness recommendation system can be implemented on a computing system. In this disclosure, the “computing system” may also be referred to as a “computing platform,” “mobile device,” “smartphone,” “laptop,” “desktop computer,” or other computing devices.

The wellness practices offered by the wellness recommendation system, also referred to as a “mobile application”, include but are not limited to guided meditations, mindfulness exercises, breathwork, sleep meditations, sound healing, music, white noise and soundscapes, stress-relief exercises, inspirational talks, physical exercises, emotional intelligence training, and spiritual guidance. These practices can be presented to the user in the form of audio and video, audio only, image only, and image and audio formats.

In one embodiment, the wellness recommender system (“Recommender”) can implement an AI-driven system that enhances user engagement and outcomes in mindfulness practices. The Recommender enables personalized recommendations based on an individual's emotional state, available time, personal goals and preferences. The Recommender can be configured to mitigate decision fatigue and choice paralysis often experienced by users when selecting appropriate mindfulness practices from a broad selection, and to provide quick and easy access to mindfulness practices.

In some embodiments, the Recommender uses an AI-driven emotional analysis module that analyzes user input which may be in the form of text or voice. The analysis module processes this input and categorizes emotions based on the Wheel of Emotion (“WoE”) framework to provide a comprehensive understanding of the user's current emotional state. In one embodiment, the system can be configured to use a large language model (LLM) to analyze the semantic content, tone, and intensity of user inputs-provided as either text or transcribed voice. The LLM need not rely on rule-based keyword detection or emotion-specific classifiers, rather, the LLM interprets natural language statements in context, using its pre-trained understanding of emotional nuance and human affective expression. The LLM can use the WoE as a reference taxonomy for categorizing user emotions. In some embodiments, the LLM can generate a natural language summary or intermediate label that reflects the perceived emotional state of the user (e.g., “anxious,” “grieving,” “overwhelmed”), which is then programmatically mapped to one of the primary, secondary, or tertiary emotion categories defined in the WoE. In certain embodiments, this approach can facilitate the system to make high-fidelity, flexible emotional classifications based on real-world language while retaining structured emotional tagging aligned with a psychologically validated model. The mapping step can be lightweight and rules-based, and can integrate seamlessly into the larger LLM-driven input analysis pipeline.

The Recommender can leverage this analysis to provide personalized recommendations by matching with a database of mindfulness practices and matching the analyzed emotional state with suitable practices. In addition to emotional state, the Recommender can also take into account user preferences and available time, ensuring that the recommended practices are not only emotionally appropriate but also practically feasible for the user.

In an exemplary aspect, the Recommender can be implemented as a software application. The user interface can be configured to allow users to describe their current emotion state. Based on this input, the Recommender automatically provides the most appropriate mindfulness practice without requiring the user to manually select from a list of options. Thus, the cognitive load on the user is significantly reduced, allowing users to engage in mindfulness practices regularly.

In some embodiments, each candidate meditation practice is associated with a relevance score calculated based on the semantic proximity of the user's input to the emotion and outcome tags stored in a database. The LLM generates this score using internal probabilistic modeling (e.g., token likelihood or attention weights), and the top N recommendations are selected by applying a threshold cutoff or top-k filtering approach. For instance, if multiple meditation practices match the user's emotional category, only those with a relevance score above a predefined threshold (e.g., 0.75) are passed through the GUI for user presentation. This thresholding reduces computation load and improves user experience by presenting only highly relevant results.

In certain embodiments, a commercially available large language model (LLM), such as the OpenAI GPT API, is used to semantically interpret user input and infer emotional context. Using the LLM, the system applies post-processing logic on the LLM's output to determine the most appropriate recommendations.

In other embodiments, the output of the LLM can be enhanced with a relevance scoring mechanism based on keyword proximity, phrase similarity, or natural language scoring returned by the model (e.g., via log probability or similarity confidence if made available by the API).

In some embodiments, the system can be configured to implement a rule-based or machine-learned threshold (e.g., only include meditations with a score above 0.75, or top 3 ranked results) to improve the quality of recommendation selection.

In certain embodiments, preferably the fixed-order prioritization acts as a constraint to avoid unnecessary computation over non-relevant records. For example, if no practices match the current emotional state, the system proceeds to desired outcome filtering, skipping subsequent tiers. Similarly, by structuring duration selection into a prioritized sequence—within-range first, then shorter, then slightly longer—the system minimizes total database queries and improves response latency by stopping as soon as a match is found in the current tier. This sequential narrowing process eliminates the need to evaluate the full record set, reducing both time and processing requirements.

In one embodiment, in operation, the system executes a processing pipeline that receives request data in the form of user-provided text or transcribed speech and applies an input-analysis module executed by an LLM engine using an NLP engine and constrained by the Wheel-of-Emotion framework to interpret the user's current affect and generate one emotional-state classification for that request. The NLP engine identifies contextually relevant phrases indicative of affect, the LLM interprets those phrases in the context of the Wheel-of-Emotion framework, and the system programmatically maps the interpretation to a single category of the WoE framework. The resulting single emotional-state classification is supplied to downstream components, including the meditation-matching module and/or the personalized recommendation module, which uses the classification in subsequent filtering and recommendation steps.

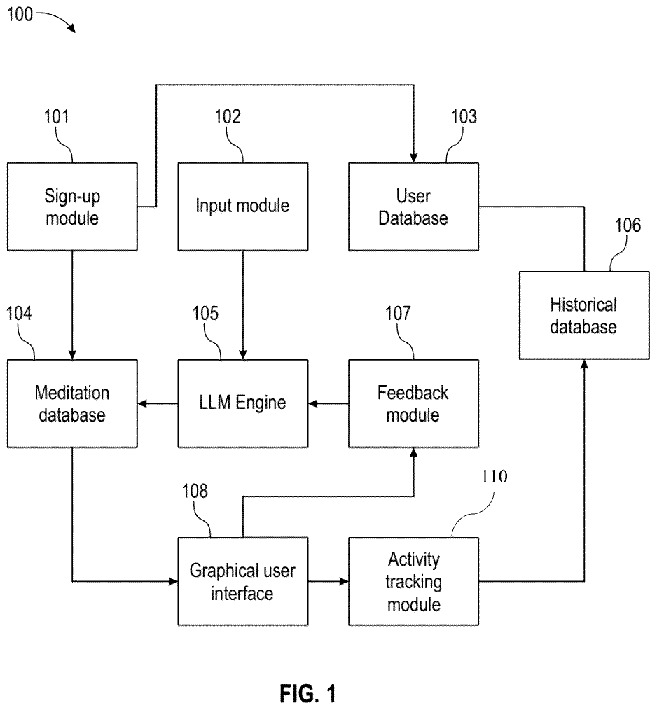

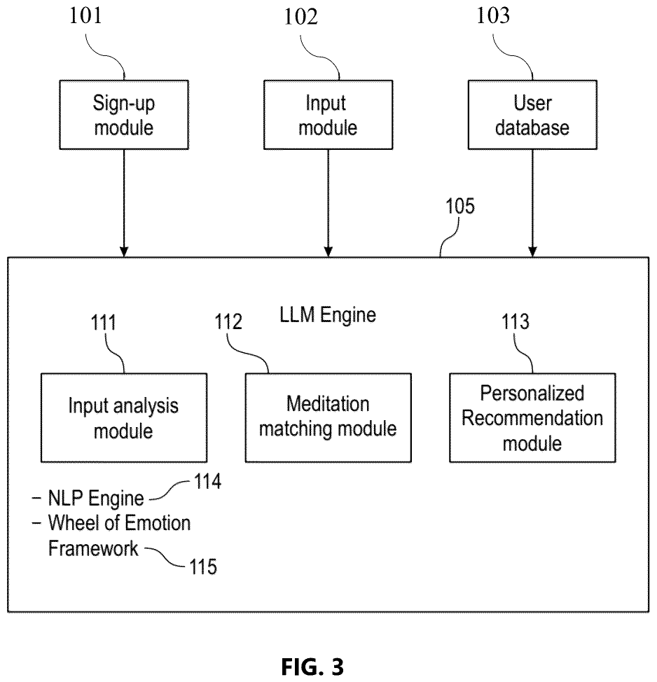

In one embodiment, a wellness recommendation system 100 can be configured as shown in the high level block diagram of FIG. 1. System 100 can be implemented as a computerized application. The system 100 can include a graphical user interface (GUI) 108, a sign-up module 101, an input module 102, a user database 103, a meditation database 104, a large language model (LLM) engine 105, a historical database 106, and a feedback module 107. In some embodiments, system 100 can include an activity tracking module 110 for facilitating the collection of data associated with the user's use of the system 100. Referring to FIG. 3, the LLM engine 105 can include an input analysis module 111, a meditation matching module 112, and a personalized meditation recommendation module 113. The sign-up module 101 receives basic details about the user including, for example, name, age, gender, city, lifestyle, interests, level of guidance preferences, wellness goals, and experience with meditation and mindfulness.

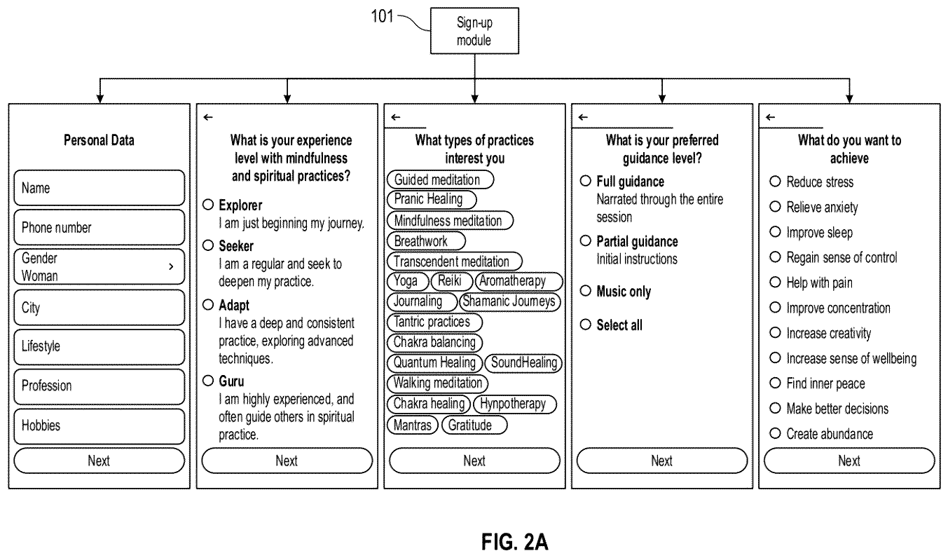

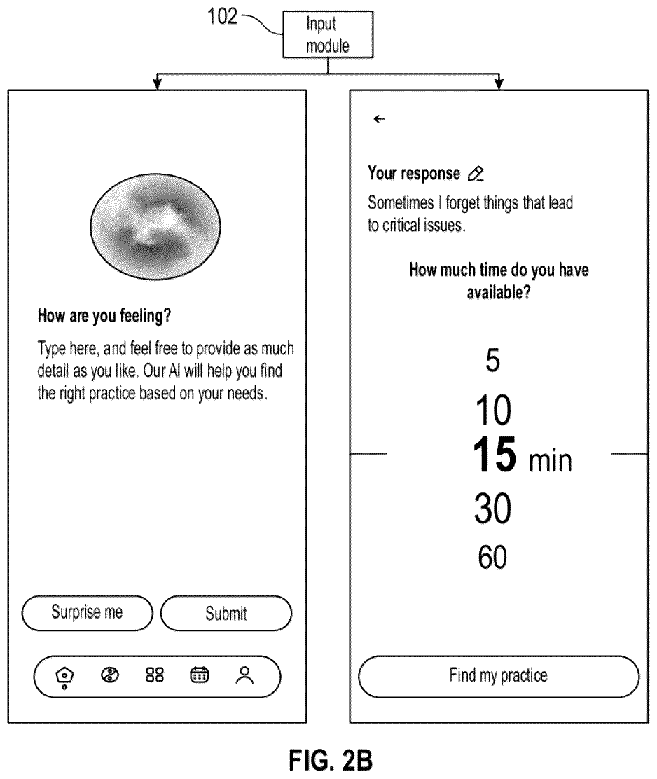

FIG. 2A depicts an exemplary GUI 108 in which the user selects/enters sign-up data via the sign-up module 101. The input module 102 receives meditation request data including real-time user data. FIG. 2B depicts an exemplary GUI 108 in which the user inputs/selects the meditation request data. The data acquired during the sign-up process are stored in the user database 103. However, the user is allowed to update the aforementioned details anytime, and the user database 103 is updated accordingly.

The meditation database 104 includes a plurality of meditation related data including but not limited to name of the meditation, description of the meditation, guidance levels, emotion states, emotional impact, practice duration, and user goals.

The meditation data in the meditation database 104 can be structured with, at least some, of the following fields with their corresponding description:

Name: Name of the meditation

Description of the meditation: This field describes the meditation and its purpose. It may be linked to a specific situation. For example, “it helps to deal with the loss of a loved one,” or “helps to manifest more abundance,” or “it is for when the user in a dilemma.” If the user input contains phrases that describe a specific situation, then the system 100 selects this meditation if similarities are found.

Wellness goals: The wellness goals include but are not limited to anxiety management, sleep improvement, overcoming addictions, improving decision making ability, promoting mindfulness, manifestation, improving emotional resilience, improving concentration, increasing self-awareness, building self-compassion, developing empathy and compassion, overcoming fear, spiritual growth, reducing anger, enhancing patience, and improving time management.

Level of experience with mindfulness: Beginner, Intermediate, Experienced, Expert (terms used to indicate these may differ). Based on user's experience level and exposure to mindfulness, wellness practices are recommended to them.

Guidance level: This field defines that the meditation is fully guided, partially guided or only music. This should be taken into account to cater to the user preferences which are provided in the user input.

User state: These are basic emotions, as defined by the WoE. The system 100 can be configured to recognize what emotional state the user is in, and to recommend meditations that are made for that emotional state. If the user states they are angry, for example, the system 100 selects meditations that are made for anger.

Emotional impact: This field defines emotional impact of a meditation. The system 100 selects meditations that have the emotional impact the user desires. For example, if the input contains phrases such as “I want something relaxing” or “I'd like to listen to something uplifting” then the system 100 chooses meditations with respective emotional impact.

Duration—This field includes the length of the meditation in minutes. In the input, the user states his available time. The system 100 can be configured to choose which meditation fits all the above criteria as well as the user's available time. In one aspect, referencing FIG. 2B, the user can choose between 1, 3, 5, 10, 15, 30, 60 minutes as wellness practice duration. In such cases, the system 100 selects meditations that are within that time frame. In another aspect, the system 100 selects meditations that match other user criteria with the duration that is closest to the duration chosen by the user. For example, if the user indicates available time of 10 minutes to do a meditation to help him calm his anger, and there is a meditation practice for releasing anger that is 11 minutes and one for 30 minutes, the system 100 can suggest the practice for 11 minutes. The system 10 can also suggest a practice that is under the indicated available time. For example, a breathing exercise that is 7 minutes long.

The process of inputting sign-up data includes user filling in basic details, answering one or more questions generated by the system 100, user selecting one or more responses from a plurality of responses for one or more questions being generated by the system 100 including meditation experience level, the type of wellness practice, guidance level, wellness goal, or combination of both.

Upon sharing the initial sign-up data, the user inputs the request data in the input module 102 for receiving wellness recommendation. The request data can include the current emotional state of the user (for example, “How are you feeling?”) and the user's time availability as shown in FIG. 2B. The request data can be provided in the form of text or voice to the input module 102. In one embodiment, the GUI 108 can be configured to request the user's current state, and another GUI 108 configured to facilitate the user to input their current state, respectively. Once the user enters the details about their current state, the user's time availability is requested as shown in FIG. 2B.

Upon receiving the user inputs, the LLM engine 105 can provide a wellness recommendation based on the user inputs such as the sign-up data and the request data. The system 100 provides a recommendation following analysis of the user inputs. The LLM can be configured to determine basic emotions (e.g., fear, disgust, anger, joy, sadness, surprise) or secondary and tertiary emotions as defined by the WoE framework. The system 100 can be configured to make the identification based on explicit emotional indicators provided by the user or by assessing the intensity and tone of the language used in the input. For instance, phrases like “feeling overwhelmed” or “seeking relaxation” can be analyzed by the system 100 to infer emotional states such as stress or a desire for calmness, while also considering both direct expressions and subtle linguistic patterns.

In an exemplary embodiment, the LLM engine 105 considers a plurality of attributes to provide appropriate meditation recommendation to the user. The plurality of attributes can be stored in the meditation database 104. For instance, the plurality of attributes include, but are not limited to, the user's present situation, for example, the name of the meditation, guidance level, user's emotional state, emotional impact, wellness goals, experience level with meditation and mindfulness, and duration (that is, the user's availability). The input analysis module 111 can include a natural language processing (NLP) engine 114 and a Wheel of Emotion (“WoE”) framework 115. In one embodiment, the input analysis module 111 analyzes the request data, by utilizing an NLP engine 114, to obtain contextual input phrases. The contextual input phrase is further utilized by the WoE framework 115 to identify in real time a likely emotional state of the user.

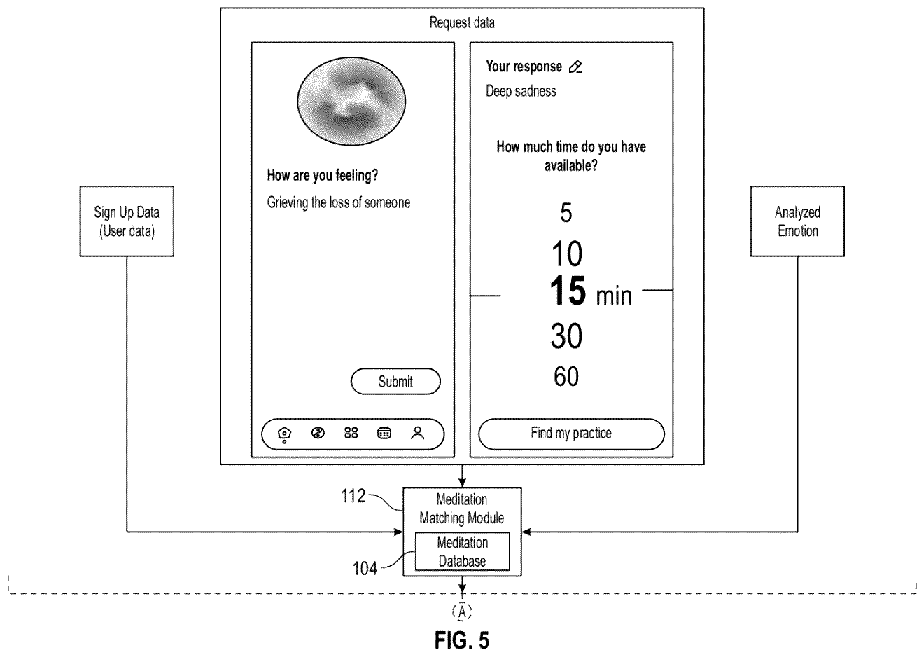

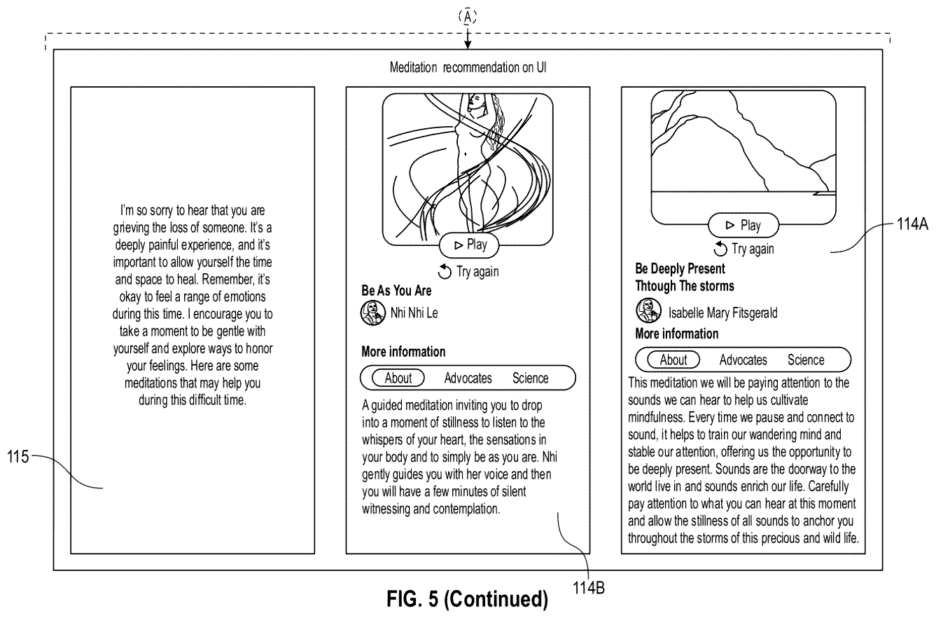

In one embodiment, upon determining user's emotional state and current situation, the system 100 displays a compassionate message 115 addressing the user's emotional state as shown in FIG. 5. This gives the user a sense of being heard and supported.

In an exemplary embodiment, the meditation matching module 112 can be configured to match the request data with one or more meditations in the meditation database 104 based on identified user's emotional state.

In some aspects, referring FIG. 5, the meditation matching module 112 utilizes user's emotional state and identifies meditations from the meditation database 104 that are associated with similar emotional states. The matching module 112 then selects appropriate meditations by evaluating the similarity between the user's emotional state and the corresponding meditations in the database 104.

When no meditation fully matches all aspects of the request data, the matching module 112 can implement hierarchical prioritization to select the meditation that most closely aligns with the user's needs. For example, the hierarchical prioritization considers multiple factors in a configurable hierarchy, with primary consideration given to the user's current emotional state, followed by their desired outcome, guidance level preferences, and available time duration. The hierarchy can be adjusted to accommodate varying user requirements and preferences.

To ensure the selected meditations accommodate within the user's available time duration, the matching module 112 performs temporal adjustments through several methods. For example, matching module 112 first attempts to identify meditations with durations that most closely match the user's indicated available time. When exact duration matches are unavailable, the module can substitute alternate versions of meditations. In cases where shorter duration alternatives cannot be found, the matching module 112 may provide slightly extended versions of appropriate meditations while staying as close as possible to the user's time availability.

In some embodiments, the meditation matching module 112 can be configured to match the user's present situation, using the phrases input by the user, with an appropriate meditation to be provided to the user. The user's present situation can be an attribute that describes the meditation and its purpose. This attribute can be linked to a specific situation. For example, this attribute “helps to deal with the loss of a loved one,” or “helps to manifest more abundance,” or “when the user in a dilemma.” If the user input contains phrases that describe a specific situation, and the meditation matching module 112 finds a meditation that corresponds to the situation, an appropriate meditation is played.

In some embodiments, the meditation matching module 112 matches the guidance level, input by the user, with respective meditation to provide meditation accordingly. For example, the guidance may be fully guided, partially guided or only music based guidance.

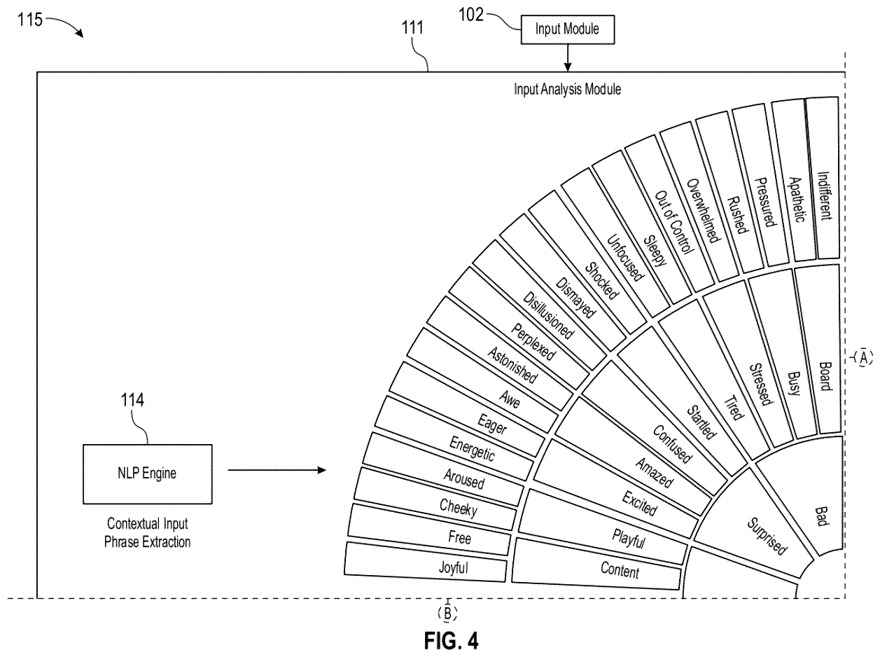

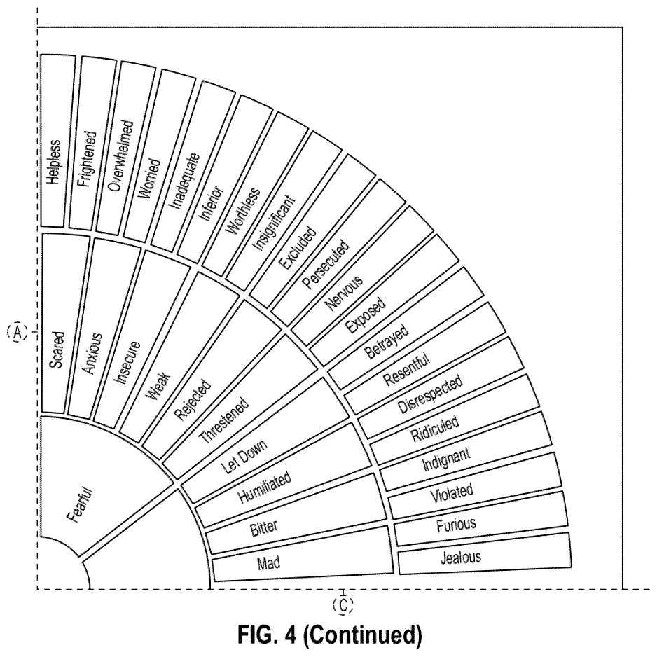

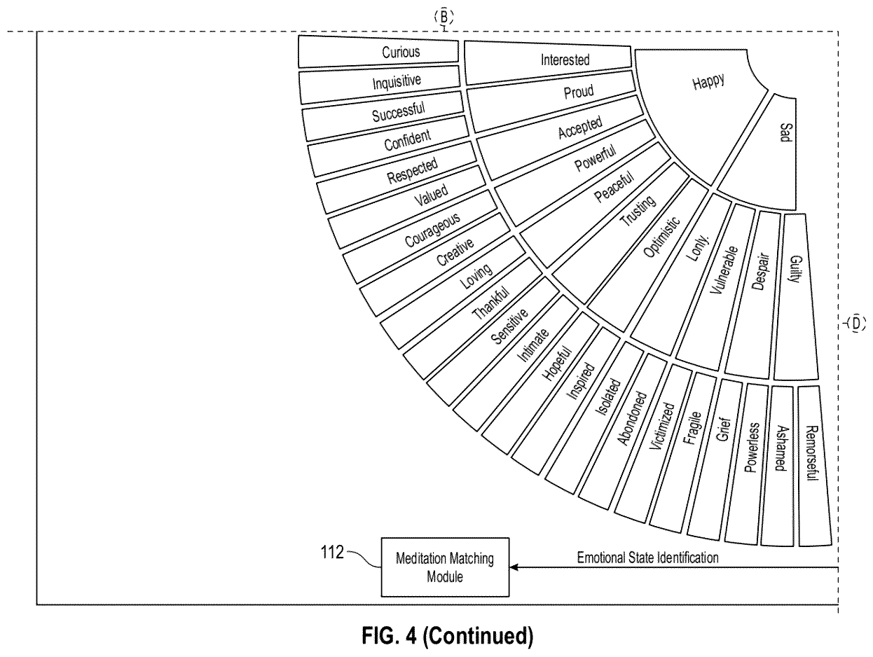

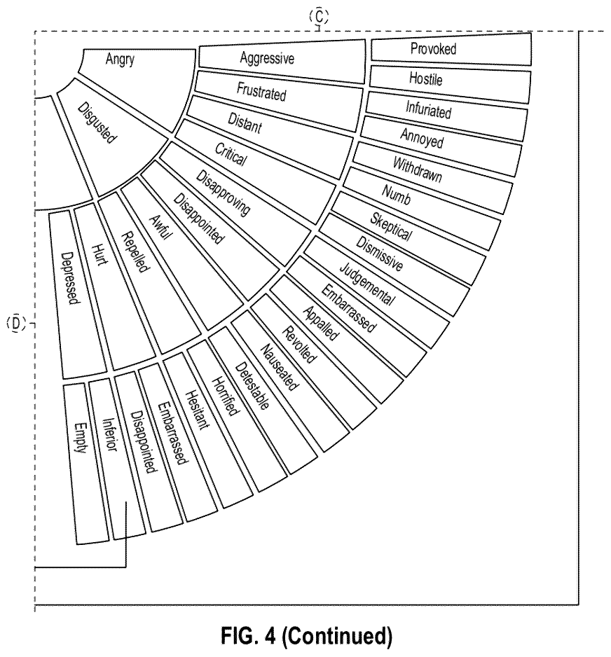

In some embodiments, the meditation matching module 112 matches the user's emotional state with the WoE framework 115 as shown in FIG. 4. Each category in the WoE framework is associated with one or more specific meditations. For example, if the user states he is angry, meditations appropriate to anger as an emotion are selected.

The meditation matching module 112 matches the emotional impact of a meditation practice, the emotional impact input by the user while signing up or later. For example, if the user input contains language such as “I want something relaxing” or “I'd like to listen to something uplifting,” then the meditation matching module 112 matches meditations based on the emotional impact input.

The meditation matching module 112 matches the duration of user's availability with the meditation of appropriate duration. The user's input before initiating a meditation session can include the duration information. The meditation matching module 112 can be configured to choose a meditation practice combining all the above criteria, and then duration. For example, the user can input time duration of their preference or choose between 1, 3, 5, 10, 15, 30, or 60 minutes as duration. The meditation matching module 112 chooses meditations that are within the chosen time frame.

The meditation matching module 112 is preferably configured to match the meditation that satisfies all the aforementioned attributes. However, if there are no meditations that fit all attributes, the matching module 112 chooses the closest meditation. The matching module 112 can be configured to prioritize practices in following hierarchy: the user's current emotional state, desired outcome/goal from the practice, the guidance level, and then the duration. The hierarchy can be changed by adjusting the settings of the system 100, and new factors may be added in the future. In some embodiments, the hierarchy can be duration, then goal of the practice, then current emotional state, then guidance level.

For example, suppose the user wants to do a meditation to help with insomnia, has 5 minutes, and prefers fully guided meditations. The meditation matching module 112 selects a meditation that matches all three criteria. However, if there is no practice that matches all three criteria, it selects the practice that matches them the closest, with priority given to, for example, the desired outcome and emotional state of the user.

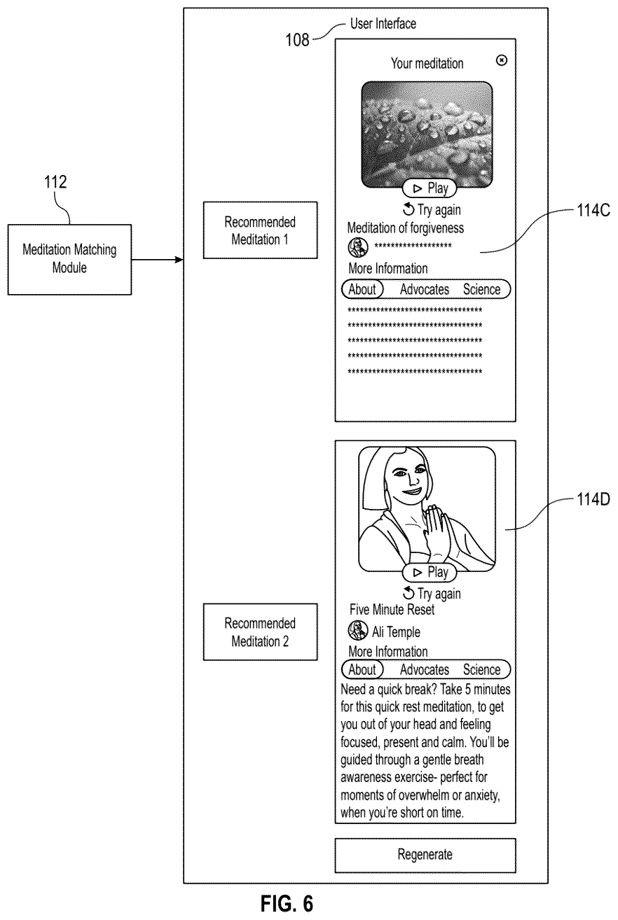

As shown in FIGS. 5 and 6, the GUI 108 can be configured to display closely matched practices as recommended meditations 114A, 114B, 114C, 114D or wellness practice as recommended by the meditation matching module 112. The GUI 108 can be configured to facilitate the user selecting at least one meditation from the recommended meditation to play on the GUI 108 and practice by them. The meditation output can be in JavaScript Object Notation (JSON) format, which can include both the compassionate or supportive message, and the selected meditation. In one embodiment, the GUI 108 can be configured to facilitate the user regenerating the meditation if the suggested meditation is not useful to the user, so that the meditation module 112 provides alternate meditation practices.

The feedback module 107 can be configured to prompt the user to rate the recommended meditations at the end of the practice. This can facilitate the future recommendations and to improve the efficiency of the Recommender.

For example, a user inputs “I'm feeling overwhelmed and stressed about my upcoming presentation.” The LLM engine 105 processes the input and identifies anxiety as the primary emotion. The meditation matching module 112 then queries the meditation database 104 for meditations known to reduce anxiety, filtering the results based on the user's available time (e.g., 10 minutes), and other indications that they are interested in breathwork techniques. The system 100 then recommends a 10-minute guided breathing exercise specifically designed for anxiety relief, which the user can start immediately.

All the activities of the user and the LLM engine 105 can be recorded, such as the individual user input, and the user response and recommendations, including the user's request refinement of suggested meditation, accepting the suggested meditation, completion of the full meditation or exiting the meditation while in progress, and the feedback rating. The aforementioned data can be stored in the historical database 106 of the system 100, and said data can be used to train the LLM engine 105 to provide more accurate responses to the user, and also fed into the personalized recommendation module 113.

In an exemplary embodiment, the personalized recommendation module 113 can analyze the data stored in historical database 106 and the basic details stored in the user database 103 to provide recommended meditations without receiving inputs from the user in real-time. The personalized recommendation module 113 can be distinct from the meditation matching module 112, as the personalized recommendation module 113 can be configured to automatically recommend practices by utilizing existing data stored in the user database 103. The personalized recommendation module 113 can be configured to analyze and provide recommendations by working in tandem with the meditation matching module 112. For example, the personalized recommendation module 113 can suggest meditations based on the emotional state of the user's last input or activities in the last 24 hours and user preferences, can suggest (about 70% of the time) practices that match user's interest and new types of practices (about 30% of the time), can suggest (about 70% of the time) meditations of user preferred guidance levels and other types of guidance levels (about 30% of time), and can suggest (about 70% of the time) practices that help user achieve goals and practices that work on other goals (about 30% of the time). The aforementioned percentages are merely exemplary, and can vary based on the preferred setting of the system 100.

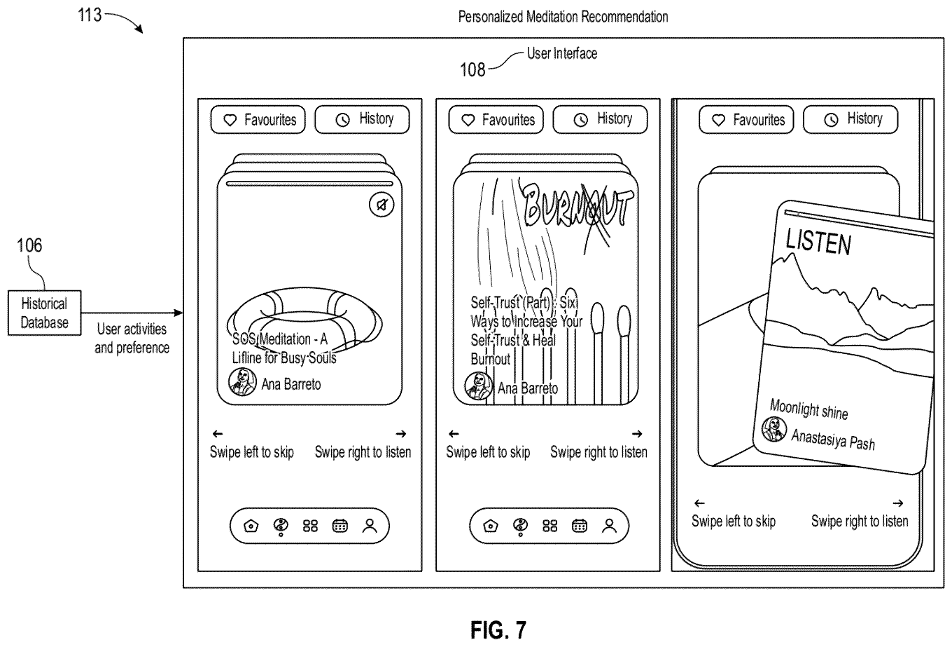

In one embodiment, the GUI 108 can be configured to facilitate the user viewing or listening to the suggested meditation on the GUI 108, and swipe the recommended meditation to left, for example, on the GUI 108 when the meditation is not of the user's interest. The user can swipe right if the suggested meditation is of the user's interest. FIG. 7 shows GUIs 108 with suggested meditations that the user can swipe right or left based on their preference to listen or skip, respectively. The user's response to the suggested meditation using the personalized recommendation module 113 can be recorded in the historical database 106 and may be shared with users of similar profiles and preferences.

While the disclosed embodiments of the invention use LLM models for emotional analysis and recommendations, other AI models or frameworks may also be potentially employed for emotional analysis, allowing for future improvements and adaptations. Additionally, the system may be enhanced to accept additional input modalities, such as biometric data (e.g., heart rate, facial expressions), to further refine the emotional analysis process.

According to an embodiment, a wellness recommendation system (“Recommender”) can a computer-implemented method for providing personalized meditation recommendations to individual users based on their emotional states, preferences, and wellness goals. The method can be implemented using the system 100 disclosed in FIG. 1. The system 100 can include a meditation database comprising a plurality of meditations, a user database, and a graphical user interface (GUI) to enhance the user's meditation experience.

In an embodiment, the method involves receiving, via the GUI 108, user data that includes information associated with the user such as name, age, gender, city, lifestyle, interests, guidance preferences, wellness goals, and meditation experience level. The user data can be stored in the user database and can serve as input for generating personalized recommendations. In an exemplary embodiment, the method can further involve capturing request data. In some embodiments, the method involves receiving request data in various formats, including text and voice inputs. Natural language processing (NLP) can be applied to process these inputs, extract key attributes, and generate contextualized recommendations. For instance, spoken phrases like “I have 10 minutes and feel stressed,” or “Today I want to manifest,” or “I'm struggling to fall asleep” can be parsed to identify relevant parameters such as time duration, emotional state, and desired practice type. In some embodiments, the request data can include parameters such as the user's current emotional state, the desired type of wellness practice, and the available time duration. The request data can be collected for aligning future recommendations with the user's needs and contextual preferences.

In some embodiments, the received request data can be analyzed by the LLM using a Wheel of Emotions (“WoE”) framework. The analysis can involve leveraging the WoE framework to identify the user's emotional state. The LLM can determine basic emotions (e.g., fear, disgust, anger, joy, sadness, surprise) or secondary and tertiary emotions as defined by the WoE framework. The identification can be based on explicit emotional indicators provided by the user or by assessing the intensity and tone of the language used in the input. For instance, phrases like “feeling overwhelmed” or “seeking relaxation” can be analyzed to infer emotional states such as stress or a desire for calmness, while also considering both direct expressions and subtle linguistic patterns.

A meditation database can store a plurality of meditations, each meditation annotated with attributes such as a meditation name, description, guidance level, emotional state association, emotional impact, duration, and associated wellness goals.

In some embodiments, the method can include matching the request data with one or more meditations in a meditation database based on the identified user's emotional state. The matching process can include analyzing contextual input phrases in the request data that describe the user's emotional state, identifying meditations associated with similar emotional states in the meditation database, and selecting meditations based on the similarity between the emotional state and the corresponding meditation from the meditation database.

The method further involves displaying the matched meditations as recommendations via the GUI. In some embodiments, the recommendations can be presented for user selection and playback. When the user selects a desired meditation among the recommended meditations, the selected meditation is displayed in the GUI along with a compassionate message addressing the user's emotional state.

In another embodiment, the method involves providing an option for the user to regenerate recommendations if the displayed meditations do not meet their expectations. In some aspects, the user feedback can be collected through the GUI and used to generate alternative recommendations. The feedback can be stored in the historical database for refining future recommendation algorithms.

In certain embodiments, when no meditation matches all the request data, the method can include selecting a meditation that most closely matches the request data based on a hierarchical prioritization. This hierarchical prioritization can include prioritizing meditations based on a configurable hierarchy that considers, at least, the user's current emotional state, the desired outcome, the guidance level, or the available time duration. The hierarchy can be configured to meet varying user needs.

In other embodiments, the selected meditations are adjustable to accommodate the user's available time duration. The adjustment process comprises at least one of: prioritizing meditations having durations most closely matching the user's indicated available time; substituting alternate versions of meditations when meditations matching the exact duration are not available; and providing slightly extended versions of meditations when shorter duration alternatives are not available.

In an exemplary embodiment, the method can involve analyzing the historical database and user data to generate automated meditation recommendations without real-time input. In one aspect, the analysis considers the user's previous emotional states, interests, preferred guidance levels, wellness goals within a predefined time period, and other user-specific parameters derived from the historical database or user data. For example, the user-specific parameters may include, but are not limited to, preferences such as male or female voices. Further, in this embodiment, the system 100 can include functionality to adapt recommendations dynamically based on swipe-based interactions in the GUI. Users can indicate interest or disinterest in specific meditations, and these responses are recorded in the historical database to refine future suggestions.

The disclosed method significantly improves the personalization and accuracy of meditation recommendations by integrating user-specific data and current emotional state analysis. In some embodiments, combination of meditation database, user feedback, and natural language processing (NLP) capabilities can yield recommendations that are contextually relevant and aligned with individual wellness goals and preferences.

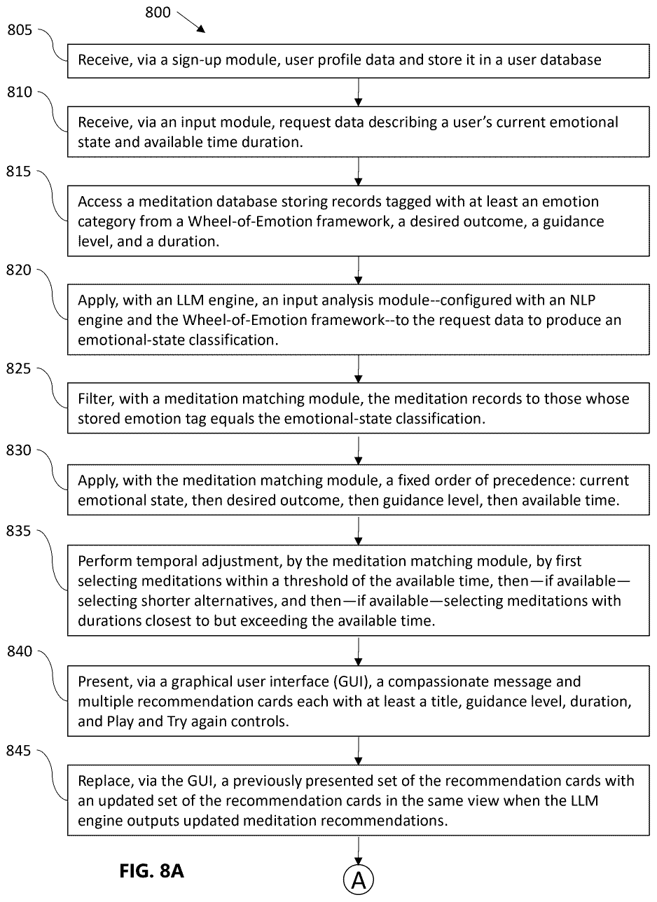

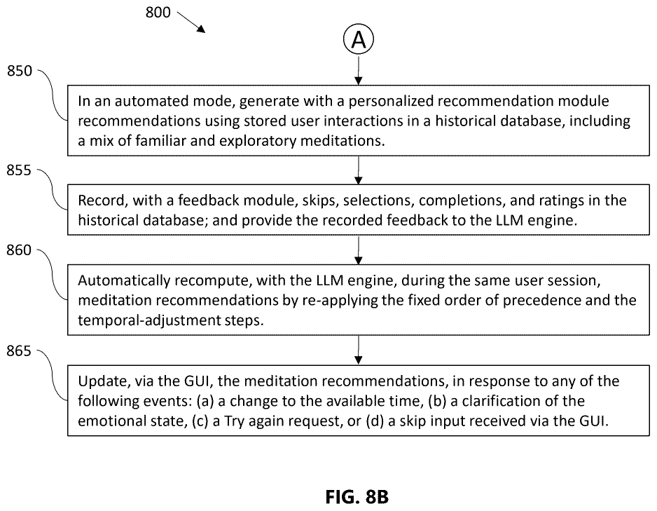

Referencing FIG. 8, method 800 of recommending a wellness practice can include receiving (805), via a sign-up module, user profile data and storing the user profile data in a user database. Method 800 can involve receiving (810), via an input module, request data (e.g., user text or voice) describing a current emotional state and an available time duration. Method 800 can include accessing (815) a meditation database storing records tagged with at least one emotion category from a Wheel-of-Emotion framework, a desired outcome, a guidance level, and a duration. Method 800 can involve applying (820), by an LLM engine, an input analysis module configured with an NLP engine and the Wheel-of-Emotion framework to the request data to produce an emotional-state classification. In one embodiment, method 800 can include filtering (825), by a meditation matching module, the meditation records to those whose stored emotion tag equals the emotional-state classification. In some embodiments, method 800 can involve applying (830), by the meditation matching module, a fixed order of precedence: current emotional state, then desired outcome, then guidance level, then available time. In certain embodiments, method 800 can further include performing temporal adjustment (835), by the meditation matching module, by first selecting meditations within a threshold of the available time, then-if available-selecting shorter alternatives, and then-if available-selecting meditations closest to but exceeding the available time.

In one embodiment, method 800 can involve presenting (840), via a graphical user interface (GUI), a compassionate message and multiple recommendation cards each with at least a title, guidance level, duration, and Play and Try again controls. In some embodiments, method 800 can include replacing (845), by the GUI, a previously presented set of the recommendation cards with an updated set of the recommendation cards in the same view when the LLM engine outputs updated meditation recommendations.

In certain embodiments, method 800 can include, in an automated mode, generating (850) with a personalized recommendation module recommendations using stored user interactions in a historical database, including a mix of familiar and exploratory meditations according to predetermined percentages. In one embodiment, method 800 can further involve recording (855), by a feedback module, skips, selections, completions, and ratings in the historical database, and providing the recorded feedback to the LLM engine.

In some embodiments, method 800 can further include automatically recomputing (860) with the LLM engine, during the same user session, meditation recommendations by re-applying the fixed order of precedence and the temporal-adjustment steps, and then with the GUI updating (865) the meditation recommendations, in response to any of the following events: (a) a change to the available time, (b) a clarification of the emotional state, (c) a Try again request, or (d) a skip input received via the GUI.

In some embodiments, system performance can be monitored based on, for example, average decision latency (time to playing a practice), completion rate of recommended practices, and user-reported satisfaction scores. The feedback module can be configured to continuously update the historical database to minimize average latency and maximize emotional engagement through adaptive filtering.

In some embodiments, a processing pipeline for implementing inventive methods of meditation recommendation can be as follows.

```
  // Step 1: Collect user input
  user_input = get_user_input_text( )
  available_time = get_user_duration_selection( ) // e.g., 5, 10, 15 mins
    // Step 2: Extract context from input using LLM
    emotion = LLM.extract_emotion(user_input)
    situation = LLM.extract_context(user_input)
    emotional_impact = LLM.extract_desired_impact(user_input)
    // Step 3: Filter meditations based on extracted parameters
    candidates = DB.filter_by({
    emotion: emotion,
    situation: situation,
    impact: emotional_impact,
    guidance_level: preferred_guidance,
    duration: match_duration(available_time)
    })
    // Step 4: If no exact match, find closest based on priority
    if candidates.is_empty( ):
    fallback_candidates = DB.find_closest_match([
    emotion, situation, duration, guidance_level
    ])
    selected = fallback_candidates.limit(3)
    else:
    selected = candidates.limit(3)
    // Step 5: Generate compassionate message
    message = LLM.generate_compassionate_response(user_input, emotion)
    // Step 6: Output as JSON
    return {
    “text”: message,
    “meditations”: join_names(selected)
    }
    In some embodiments, automated meditation recommendations can be
implemented as follows.
    // Step 1: Load user data
    user_requests = get_recent_user_requests( ) // Freeform user input
    user_preferences = get_user_preferences( ) // Guidance level, emotional needs,
goals
    // Step 2: Analyze emotional state and situation
    detected_emotion = LLM.extract_emotional_state(user_requests)
    detected_situation = LLM.extract_contextual_situation(user_requests)
    desired_impact = LLM.extract_desired_emotional_impact(user_requests)
    user_goal = user_preferences.goal
    preferred_guidance = user_preferences.guidance_level
    // Step 3: Filter meditations based on emotional state and situation
    if user_requests:
    emotion_matched = DB.filter(user_state = detected_emotion)
    situation_matched = DB.search_about_field(similar_to = detected_situation)
    else:
    emotion_matched = [ ]
    situation_matched = [ ]
    // Step 4: Apply weighted selection logic
    // 70% from previously liked types (interests/goals/guidance), 30% exploratory
    interest_matched = DB.filter(user_goal = user_goal)
    guidance_matched = DB.filter(guidance_level = preferred_guidance)
    exploratory_interest = DB.exclude(user_goal = user_goal)
    exploratory_guidance = DB.exclude(guidance_level = preferred_guidance)
    // Emotional impact matching (e.g., calming, uplifting)
    impact_matched = DB.filter(emotional_impact = desired_impact)
    // Step 5: Aggregate results with weighted sampling
    candidates = merge_weighted({
    “emotion_matched”: 0.3,
    “situation_matched”: 0.3,
    “interest_matched”: 0.25,
    “guidance_matched”: 0.25,
    “impact_matched”: 0.3,
    “exploratory_interest”: 0.1,
    “exploratory_guidance”: 0.1
    })
    // Step 6: Score and select best matches
    ranked = rank_by_multi_match_score(candidates, criteria=[
    detected_emotion, detected_situation, user_goal,
    preferred_guidance, desired_impact
    ])
    final_recommendations = ranked.top(min=15, max=30)
    // Step 7: Format output as JSON
    return {
    “meditations”: join_names(final_recommendations)
    }
```

In an embodiment, a computer system can be configured to implement the method disclosed herein. A computer system can be programmed or otherwise configured to operate the meditation recommendation program (MRP). The computer system can regulate various aspects of the MRP such as the system that includes a sign-up module, a user database, a meditation database, an input module, a large language model (LLM) module, a feedback module, and a historical database. The computer system can be an electronic device of a user or a computer system that is remotely located with respect to the user's electronic device. The user's electronic device can be a mobile electronic device.

The computer system can include a central processing unit (CPU, also “processor” and “computer processor” herein), which can be a single-core or multi-core processor, or a plurality of processors for parallel processing. The computer system also includes memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage, and/or electronic display adapters. The memory, storage unit, interface, and peripheral devices are in communication with the CPU through a communication bus (solid lines), such as a motherboard. The storage unit can be a data storage unit (or data repository) for storing data. The computer system can be operatively coupled to a computer network (“network”) with the aid of the communication interface. The network can be the Internet, an intranet and/or extranet, or an intranet and/or extranet that is in communication with the Internet. The network in some cases is a telecommunication and/or data network. The network can include one or more computer servers, which can enable distributed computing, such as cloud computing. The network, in some cases with the aid of the computer system, can implement a peer-to-peer network, which may enable devices coupled to the computer system to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system can be included in the circuit. In some cases, the circuit is an application-specific integrated circuit (ASIC).

The storage unit can store files, such as drivers, libraries, and saved programs. The storage unit can store user data, e.g., user preferences and user programs. The computer system in some cases can include one or more additional data storage units that are external to the computer system, such as those located on a remote server in communication with the computer system through an intranet or the Internet.

The computer system can communicate with one or more remote computer systems through the network. For instance, the computer system can communicate with a remote computer system of a user. Examples of remote computer systems include personal computers (e.g., portable PC), slate or tablet PCs (e.g., Apple® iPad, Samsung® Galaxy Tab), telephones and smartphones (e.g., Apple® iphone, Android-enabled devices). The user can access the computer system via the network through various smart devices including but not limited to wearable computing devices (e.g., smartwatches, fitness trackers, smart rings), extended reality (XR) devices (e.g., smart glasses, virtual reality headsets, augmented reality devices), smart home devices and IoT (Internet of Things) controllers, portable health monitoring devices and wellness-related smart devices, convertible and hybrid computing devices that combine multiple form factors, smart automotive systems and vehicle-integrated computing devices. The remote computing systems can interface with the computer system through various networking protocols, including cellular networks (5G, LTE), Wi-Fi, Bluetooth, NFC (Near Field Communication), and other wireless communication standards. The devices may implement various security protocols and authentication methods to ensure secure access to the computer system. The remote computing systems may operate on various platforms and operating systems, including but not limited to iOS, Android, Windows, Linux, and custom embedded operating systems, while maintaining compatibility with the computer system through standardized APIs and communication protocols.

Methods as described herein can be implemented by way of machine (e.g., computer processor) executable code stored on an electronic storage location of the computer system, such as, for example, on the memory or electronic storage unit. The machine-executable or machine-readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processor adapted to execute the code or can be compiled during runtime. The code can be supplied in a programming language selected to enable the code to execute in a pre-compiled or as-compiled fashion.

Aspects of the systems and methods provided herein, such as the computer system, can be embodied in programming. Various aspects of the technology may be understood as “products” or “articles of manufacture,” typically in the form of machine (or processor) executable code and/or associated data that are carried on or embodied in a type of machine-readable medium. Machine-executable code can be stored on electronic storage units, including both local storage (e.g., read-only memory, random-access memory, flash memory, hard disks) and cloud-based storage systems. “Storage” type media can include any or all of the tangible memory of the computers, processors, or the like, or associated modules thereof, such as various semiconductor memories, tape drives, disk drives, and distributed cloud storage systems. Cloud storage implementations may include public clouds, private clouds, hybrid clouds, or multi-cloud architectures, which provide scalable, non-transitory storage for the software programming. The cloud storage systems may implement redundancy, data replication, and geographic distribution of data across multiple data centers to ensure high availability and disaster recovery capabilities. All or part of the software may at times be communicated through the Internet, cloud networks, or various other telecommunication networks. Such communications, for example, may enable loading of the software from cloud storage services into local computer platforms, thereby enabling on-demand access to the application from any connected device. Software components may be distributed across cloud regions and zones. The physical and virtual elements that carry such data include optical, electrical, and electromagnetic waves, such as those used across physical interfaces between local devices, through wired and optical landline networks, cloud infrastructure networks, and over various air-links. The physical elements that carry such waves, such as wired or wireless links, optical links, cloud networking infrastructure, or the like, also may be considered as media bearing the software. As used herein, unless restricted to non-transitory, tangible “storage” media, terms such as computer or machine “readable medium” refer to any medium that participates in providing instructions to a processor for execution, including cloud-based storage and processing resources. Data synchronization between cloud storage and local caches ensures consistent application state across multiple access points while maintaining security through encryption at rest and in transit.

Hence, a machine-readable medium, such as computer-executable code, may take many forms, including but not limited to a tangible storage medium, a carrier wave medium, or a physical transmission medium. Non-volatile storage media include, for example, optical or magnetic disks, such as any of the storage devices in any computer(s) or the like, such as may be used to implement the databases, etc., shown in the drawings. Volatile storage media include dynamic memory, such as the main memory of such a computer platform. Tangible transmission media include coaxial cables, copper wire, and fiber optics, including the wires that comprise a bus within a computer system. Carrier-wave transmission media may take the form of electric or electromagnetic signals or acoustic or light waves, such as those generated during radio frequency (RF) and infrared (IR) data communications. Common forms of computer-readable media, therefore, include, for example: a floppy disk, a flexible disk, a hard disk, magnetic tape, any other magnetic medium, a CD-ROM, DVD, or DVD-ROM, any other optical medium, punch cards, paper tape, any other physical storage medium with patterns of holes, a RAM, a ROM, a PROM, an EPROM, a FLASH-EPROM, any other memory chip or cartridge, a carrier wave transporting data or instructions, cables or links transporting such a carrier wave, or any other medium from which a computer may read programming code and/or data. Many of these forms of computer-readable media may be involved in carrying one or more sequences of one or more instructions to a processor for execution.

The computer system can include or be in communication with an electronic display that comprises a user interface (UI) for providing, for example, one or more meditation recommendations. Examples of UIs include, without limitation, a graphical user interface (GUI) and web-based user interface.

1. A method for providing meditation recommendations, the method comprising:
receiving, via a sign-up module, user profile data and storing the user profile data in a user database;
receiving, via an input module, request data comprising user text or voice describing a current emotional state and an available time duration;
accessing a meditation database storing records tagged with at least one emotion category from a Wheel-of-Emotion framework, a desired outcome, a guidance level, and a duration;
executing a processing pipeline in which an LLM engine applies an input analysis module, using an NLP engine and the Wheel-of-Emotion framework, to the request data to generate one emotional-state classification;
filtering, by a meditation matching module, the meditation records to those whose stored emotion tag equals the emotional-state classification;
applying, by the meditation matching module, a fixed order of precedence: current emotional state, then desired outcome, then guidance level, then available time;
performing temporal adjustment, by the meditation matching module, by first selecting meditations within a threshold of the available time, then—if available—selecting shorter alternatives, and then—if available—selecting meditations closest to but exceeding the available time;
presenting, via a graphical user interface (GUI), a compassionate message and multiple recommendation cards each with at least a title, guidance level, duration, and Play and Try again controls;
replacing, by the GUI, a previously presented set of the recommendation cards with an updated set of the recommendation cards in the same view when the LLM engine outputs updated recommendation data;
in an automated mode, generating, by a personalized recommendation module, recommendations using stored user interactions in a historical database, including a mix of familiar and exploratory meditations according to predetermined percentages;
recording, by a feedback module, skips, selections, completions, and ratings in the historical database and providing the recorded feedback to the LLM engine; and
responsive to any of the following events: (a) a change to the available time, (b) a clarification of the emotional state, (c) a Try again request, or (d) a skip input received via the GUI, automatically, during the same user session, recomputing, by the LLM engine, recommendations by re-applying the fixed order of precedence and the temporal-adjustment steps, and updating the recommendations presented on the GUI.

2. The method of claim 1, wherein the stored records are further tagged with a meditation name, a meditation description, an emotional impact, and associated wellness goals.

3. The method of claim 1, further comprising in the automated mode generating, by the personalized recommendation module without real-time user input, recommendations based on: the user's previous emotional states within a predetermined time period, the user's interests, the user's preference for guidance, the user's wellness goals, and other user-specific parameters derived from the historical database or user data.

4. The method of claim 1, further comprising: receiving, by the GUI, a user swipe-based interaction to allow the user to indicate interest or disinterest in personalized meditation recommendations; and recording, by an activity tracking module, user responses to the personalized recommendations in the historical database for future analysis.

5. The method of claim 1, further comprising processing the request data, by the LLM engine, using natural language processing to extract the contextual input phrases such as the attributes for recommendation generation.

6. The method of claim 1, wherein analyzing the request data comprises: using a large language model to semantically interpret user input, generate a descriptive emotional label, and programmatically map the label to a predefined emotion category of the Wheel-of-Emotion framework.

7. The method of claim 1, wherein the recommendation module is configured to apply a weighted selection strategy to select a first proportion of meditation practices matching the user's prior interests, guidance level, goals, or emotional state, and a second proportion of meditation practices that differ from prior selections, such that the combined set of recommendations includes both familiar and exploratory content.

8. A meditation recommendation system comprising:
a sign-up module to receive user profile data;
a user database configured to store the user profile data;
an input module to receive request data comprising user text or voice describing a current emotional state and an available time;
a meditation database storing records tagged with at least one emotion category from a Wheel-of-Emotion taxonomy, a desired outcome, a guidance level, and a duration;
an LLM engine comprising:
an input analysis module configured to apply an NLP engine and the Wheel-of-Emotion taxonomy to the request data to produce an emotional-state classification; and
a meditation matching module configured to:
(i) filter meditations to those whose stored emotion tag equals the emotional-state classification;
(ii) apply a fixed order of precedence: current emotional state, then desired outcome, then guidance level, then available time, and
(iii) perform temporal adjustment by first selecting meditations within a threshold of the available time, then—if available-shorter alternatives, and then—if available-meditations closest to but exceeding the available time;
a graphical user interface (“GUI”) configured to present a compassionate message and multiple recommendation cards each with at least a title, guidance level, duration, and Play and Try again controls;
wherein the GUI replaces a previously presented set of the recommendation cards with an updated set of the recommendation cards in the same view when the LLM engine outputs updated recommendation data;
a historical database;
a personalized recommendation module configured to generate automated recommendations using user interactions stored in the historical database; and
a feedback module configured to record skips, selections, completions, and ratings in the historical database and to provide the feedback to the LLM engine;
wherein, responsive to any of the following events (a) a change to the available time, (b) a clarification of the emotional state, (c) a Try again request, or (d) a skip input received via the GUI, the LLM engine automatically, during the same user session, recomputes recommendations by re-applying the prioritization order and the temporal-adjustment steps and updates the recommendations presented on the GUI.

9. The system of claim 8, wherein the records stored in the meditation database are further tagged with an emotional impact, a practice duration, and associated wellness goals.

10. The system of claim 8, wherein the personalized recommendation module is further configured to generate recommendations based on a mix of familiar and exploratory meditations according to predetermined percentages.

11. The system of claim 8, wherein the LLM is further configured to analyze the historical database, and the user data; and to automatically generate personalized meditation recommendations without real-time user input based on: the user's previous emotional states within a predetermined time period, the user's interests, the user's preference for guidance, the user's wellness goals, and other user-specific parameters derived from the historical database or user data.

12. The system of claim 11, wherein the GUI is further configured to facilitate swipe-based interaction by the user to indicate interest or disinterest in personalized meditation recommendations; and the system further comprising an activity tracking module configured to record user responses to the personalized meditation recommendations in the historical database for future analysis.

13. The system of claim 8, wherein the input analysis module is further configured to process the request data using natural language processing to extract the contextual input phrases such as the attributes for recommendation generation.

14. The system of claim 8, wherein the input analysis module is configured to use the LLM to: interpret user input, generate a descriptive emotion label, and map the emotion label to a corresponding emotion category of the Wheel-of-Emotion framework.

15. The system of claim 8, wherein the personalized recommendation module is configured to generate a weighted set of recommendations comprising a majority portion selected from previously preferred meditation characteristics and a minority portion selected from meditation practices differing from the user's historical preferences, based on a predetermined weight distribution.

16. The system of claim 8, wherein the personalized recommendation module is further configured to:
receive a natural language input from a user;
analyze the input using the LLM to determine the user's emotional state, a contextual situation, and one or more preferences relating to emotional impact, guidance level, or time available;
filter the meditation database to identify candidate practices matching the extracted parameters;
implement a fallback hierarchy that selects meditations based on a priority order of emotional relevance, duration fit, and then guidance level;
generate a compassion-oriented message aligned with the user's inferred emotional state; and
return a structured output comprising the generated message and no more than three recommended meditation practices.

17. A method of providing meditation recommendations, the method comprising:
receiving a natural language user input;
analyzing the input using a large language model (LLM) to extract an emotional state, a contextual situation, and at least one preference selected from: desired emotional impact, guidance level, or duration;
identifying a set of meditation practices from a database that match at least one or more of the extracted parameters;
applying a fallback prioritization logic if no exact match is found, wherein emotional state and duration are prioritized over guidance level;
generating a personalized text message responsive to the extracted emotional state; and
returning an output comprising the generated message and a list of one or more selected meditation practices.

18. The method of claim 17, wherein the LLM is constrained to use the Wheel of Emotion framework to extract the emotional state.

19. The method of claim 17, wherein the LLM is configured to use, at least in part, an NLP engine to extract the emotional state.

20. The method of claim 17, wherein identifying a set of meditation practices further comprises using, at least in part, a historical database having information associated with previous meditation recommendations to the user.
